(19) United States
(12) Patent Application Publication
(10) Pub. No.: US 2010/0062057 A1
(43) Pub. Date: Mar. 11, 2010

(54) Formulation

(75) Inventors: Gunnar BERGE, Oslo (NO); Svein Olaf Hustvedt, Oslo (NO); Thomas Andersen, Oslo (NO)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Pronova BioPharma Norge AS.

(21) Appl. No.: 12/207,824

(22) Filed: Sep. 10, 2008

(51) Int. Cl. A61K 9/66

(52) U.S. Cl. 424/455

(57) ABSTRACT

Novel capsules comprising a marine oil in an outer shell comprising alginate are disclosed. Also disclosed are methods of preparing the novel capsules and uses of thereof.

New seamless capsules comprising at least one oily phase that comprises at least one marine oil and at least one surfactant in an alginate capsule formulation, methods of preparing the same, and uses of thereof are disclosed herein.

Compositions comprising at least one oily phase comprising at least one marine oil encapsulated in an alginate outer surface shell are disclosed. The compositions may be seamless capsules with a shell that is thinner compared to the gelatin capsules known in the art, thereby allowing a larger amount of material to be encapsulated. The at least one marine oil may thus be administered to a subject for therapeutic treatment and/or regulation of at least one health problem including, for example, irregular plasma lipid levels, cardiovascular functions, immune functions, visual functions, insulin action, neuronal development, hypertriglyceridemia, heart failure, and post myocardial infarction (MI).

In humans, cholesterol and triglycerides are part of lipoprotein complexes in the bloodstream and can be separated via ultracentrifugation into high-density lipoprotein (HDL), intermediate-density lipoprotein (IDL), low-density lipoprotein (LDL), and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Furthermore, cardiovascular morbidity and mortality in humans can vary directly with the level of total-C and LDL-C and inversely with the level of HDL-C. In addition, researchers have found that non-HDL cholesterol is an important indicator of hypertriglyceridemia, vascular disease, atherosclerotic disease, and related conditions. In fact, recently non-HDL cholesterol reduction has been specified as a treatment objective in NCEP ATP III.

Omega-3 fatty acids may regulate plasma lipid levels, cardiovascular and immune functions, insulin action, and neuronal development, and visual function. Marine oils, also commonly referred to as fish oils, are a source of omega-3 fatty acids, including eicosapentaenoic acid (EPA) and docosahexaenoic acid (DHA), that have been found to regulate lipid metabolism. Omega-3 fatty acids may have beneficial effects on the risk factors for cardiovascular diseases, for example hypertension and hypertriglyceridemia, and on the coagulation factor VII phospholipid complex activity. Omega-3 fatty acids may also lower serum triglycerides, increase serum HDL cholesterol, lower systolic and diastolic blood pressure and/or pulse rate, and may lower the activity of the blood coagulation factor VII-phospholipid complex. Further, omega-3 fatty acids seem to be well tolerated, without giving rise to any severe side effects.

One form of omega-3 fatty acid is a concentrate of omega-3, long chain, polyunsaturated fatty acids from fish oil containing DHA and EPA and is sold under the trademark Lovaza™, formerly known as Omacor® See, for example, in U.S. Pat. Nos. 5,502,077, 5,656,667 and 5,698,594. Lovaza™ comprises at least 80% by weight of omega-3-fatty acids, salts or derivatives thereof, wherein (all-Z)-5,8,11,14,17-eicosapentaenoic acid (EPA) and (all-Z)-4,7,10,13,16,19-docosahexaenoic acid comprises at least 75% by weight of the total fatty acids. In particular, each 900 mg capsule of Lovaza™ contains at least 90% omega-3 ethyl ester fatty acids (84% EPA/DHA); approximately 465 mg EPA (eicosapentaenoic acid) ethyl ester and approximately 375 mg DHA (docosahexaenoic acid) ethyl ester.

The formulation of drugs into capsules, for example, soft or hard gelatin capsules, has been reported to solve many problems associated with tablets. Stability has generally improved through the use of gelatin capsules, most notably with active pharmaceutical ingredients (APIs) which are highly susceptible to oxidation and hydrolysis. An example is vitamin A which is relatively unstable in air and light; however, when encapsulated, the contents show no significant loss of potency for 3 years or longer when stored and packaged under prescribed conditions of temperature and humidity. U.S. Patent Application Publication No. 2004/0224020 discloses an oral dosage form with active agents in controlled cores and in immediate release gelatin capsule coats.

Alginate capsules offer several benefits over gelatin capsules known in the art. For example, alginate capsules may be more temperature-stable and humidity-stable than gelatin capsules. Furthermore, alginate capsules do not require testing for bovine spongiform encephalopathy (SSE) as gelatin capsules do, and alginate capsules may decrease gastrointestinal reflux disease symptoms, such as burping. In addition, alginate capsules may be smaller due to a thinner capsule wall. A thinner wall may allow for increased fill volume for the same capsule size. Increased fill volume may result in a greater dosage per capsule, such that a subject would require fewer capsules per day for a given dose. Alginate capsules may be less sticky, such that they would be easier to swallow and not stick together. The capsules may also be clear and colorless in appearance, which may improve the perception to patients.

Alginate capsule formulations have already been reported. For example, FR 2 745 979 discloses alginate capsules comprising omega-3 fatty acids as animal feed additives. Further, for example, HU 2 030 38 discloses encapsulation of unsaturated fatty acids, fatty acid esters, and their mixtures using alginated gel.

In some instances, it may be desirable to provide a time-released delay of a pharmaceutical composition upon administration to a subject. Several references disclose enteric capsules containing omega-3 fatty acids. For example, U.S. Pat. No. 6,531,150 discloses enteric capsules having a buffer layer of a water-soluble gel containing an acid or acid salt between the content of omega-3 fatty acids and the gelatin-based coating layer. Further, for example, European Patent Application No. EP1529524 and German Application No. DE19930030 disclose gelatin capsules containing omega-3 fatty acids coated with xylose to provide resistance to gastric juice and increase stability. In addition, Belluzi et al., N. Eng. J. Med., 334(24):1557-60, 1996, and Belluzi et al., Gastroenterology, 102(4) pt.2: A542, 1992, each disclose enteric coated fish oil capsules (PUREPA®) Tillotts-Pharma) for delayed delivery.

It is to be understood that both the foregoing general description and the following detailed description are exemplary and explanatory only and are not restrictive of the invention, as claimed.

### BRIEF DESCRIPTION OF THE DRAWINGS

FIG. 1 shows average plasma concentration versus time curves of EPA and DHA after single oral dose of Omacor® and compositions of the present disclosure comprising K85EE in male minipigs: (a) average EPA plasma concentration after oral dosing of 2 g (2 capsules); (b) average DHA plasma concentration after oral dosing of 2 g (2 capsules); (c) average EPA plasma concentration after oral dosing of 4 g (4 capsules); (d) average DHA plasma concentration after oral dosing of 4 g (4 capsules).

### SUMMARY OF THE INVENTION

As used herein, the term “omega-3 fatty acids” includes natural or synthetic omega-3 fatty acids, as well as pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors, salts, and mixtures thereof. Examples of omega-3 fatty acid oils include, but are not limited to, omega-3 polyunsaturated, long-chain fatty acids such as a eicosapentaenoic acid (EPA), docosahexaenoic acid (DHA), and□-linolenic acid; esters of omega-3 fatty acids with glycerol such as mono-, di- and triglycerides; and esters of the omega-3 fatty acids and a primary, secondary and/or tertiary alcohol, such as, for example. fatty acid methyl esters and fatty acid ethyl esters. Preferred omega-3 fatty acid oils are long-chain fatty acids, for example, EPA and DHA, triglycerides thereof, ethyl esters thereof, and/or mixtures thereof. The omega-3 fatty acids, their esters, derivatives, conjugates, precursors, salts and/or mixtures thereof can be used in their pure form and/or as a component of an oil, for example, as marine oil, for example fish oil, such as purified fish oil concentrates. Commercial examples of omega-3 fatty acids s include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE, and EPAX7010EE.

In at least one embodiment, omega-3 fatty acids are chosen from Lovaza™ (formerly Omacor®/), K85, and AGP-103. According to another embodiment of the present disclosure, omega-3 fatty acids are esterified, such as alkyl esters. Those alkyl esters include, but are not limited to ethyl, methyl, propyl, and butyl esters, and mixtures thereof. In at least one embodiment, the omega-3 fatty acids are present in the form of free fatty acids.

According to another embodiment, the omega-3 fatty acids may chosen from mono-, di-, and triglycerides. In another embodiment, the omega-3 fatty acids are in the form of a phospholipid. The omega-3 fatty acids according to the present disclosure may derive from animal oils, such as marine oil, such as fish oil, krill oil, or lipid composition derived from fish. In one embodiment, the oil is a an active pharmaceutical ingredient (API). In other embodiments, the oil is a nutritional supplement. In yet other embodiments, the oil is a flavor oil, a food, and/or a food additive. Said oil may also be a carrier for oil-soluble active materials, wherein said oil-soluble active material comprises another pharmaceutical agent, nutritional agent, flavor, fragrance, or a food.

As used herein, “alginate” includes alginic acid and/or pharmaceutically acceptable salts thereof, and refers generally to a copolymer comprising (1-4)-linked β-D-mannuronate (M) and its C-5 epimer α-L-guluronate (G) residues. Non-limiting examples of alginate salts suitable for the disclosure herein include alginate salts of calcium, strontium, barium, or aluminum. In one embodiment, alginate comprises all or in part M-alginate. In another embodiment, alginate comprises all or in part G-alginate. In another embodiment, alginate comprises a combination of M-alginate and G-alginate. In at least one embodiment, the alginate has a G content of at least 30% by weight. In other embodiments, the alginate has a content ranging from about 40% to about 80% by weight.

In at least one embodiment, the alginate shell achieves a time-release delivery of omega-3 fatty acids upon administration to a subject.

In some embodiments of the present disclosure, the alginate shell further comprises coloring agents, stabilizers, sweetening agents, plasticizers, and/or hardeners.

Other polymers contemplated as comprising the capsule shell include polyesters, polyacrylates, polycyanoacrylates, polysaccharides, polyethylene glycol, and mixtures thereof. Other polymers may include, for example, gelatin, carboxymethylcellulose alginates, carrageenans, pectins, ethyl cellulose hydroxypropyl methylcellulose, cellulose acetophthalate, hydroxypropyl methylcellulose phthalate, methylacrylicacid copolymers (Eudragit® Land S), dimethylaminoethylmethacrylate copolymers (Eudragit E), trimethylammoniumethylmethacrylate copolymers (e.g., Eudragit® RL and RS), polymers and copolymers of lactic and glycolic acids, and mixtures thereof. In one embodiment, the polymer comprises a plasticizer additive, such as, for example, but not limited to, triethyl citrate, butyl phthalate, and mixtures thereof. Other additives may optionally be incorporated to improve and/or facilitate the encapsulation process, such as, for example, fluidizing agents, such as talc.

The seamless capsules of the present disclosure may comprise at least one non-active pharmaceutical ingredient (also known generally herein as “excipients”). Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Thus, the at least one non-active ingredient may include be chosen from colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide, and xanthum gum.

Surfactants may be chosen from, for example, glycerol acetates and acetylated glycerol fatty acid esters, such as acetin, diacetin, triacetin, and/or mixtures thereof. Suitable acetylated glycerol fatty acid esters include, but are not limited to, acetylated monoglycerides, acetylated diglycerides, and/or mixtures thereof.

In addition, the surfactant may be chosen from glycerol fatty acid esters, such as, for example, those comprising a fatty acid component of about 6-22 carbon atoms. Glycerol fatty acid esters can chosen from monoglycerides, diglycerides, triglycerides, and/or mixtures thereof. Suitable glycerol fatty acid esters include monoglycerides, diglycerides, medium chain triglycerides with fatty acids having about 6-12 carbons, and/or mixtures thereof. Capmul® MCM (medium chain mono- and di-glycerides) is an example.

The at least one surfactant may be chosen from propylene glycol esters. For example, propylene glycol esters include, but are not limited to, propylene carbonate, propylene glycol monoacetate, propylene glycol diacetate, propylene glycol fatty acid esters, acetylated propylene glycol fatty acid esters, propylene glycol fatty acid monoesters, propylene glycol fatty acid diesters, and mixtures thereof. Fatty acids may comprise, for example, about 6-22 carbon atoms. Examples of propylene glycol esters include, but are not limited to, propylene glycol monocaprylate (Capryol®), propylene glycol dicaprylate, propylene glycol dicaprate, propylene glycol dicaprylate/dicaprate, and mixtures thereof.

The at least one surfactant may be chosen from ethylene glycol esters, such as, for example, monoethylene glycol monoacetates, diethylene glycol esters, polyethylene glycol esters, and mixtures thereof. Additional examples include ethylene glycol monoacetates, ethylene glycol diacetates, ethylene glycol fatty acid monoesters, ethylene glycol fatty acid diesters, and mixtures thereof. In addition, the ethylene glycol esters may be chosen from polyethylene glycol fatty acid monoesters, polyethylene glycol fatty acid diesters, and mixtures thereof. Ethylene glycol esters may be obtained from the transesterification of polyethylene glycol and a triglyceride, a vegetable oil, and/or mixture thereof, and include, for example, those marketed as Labrafil® and Labrasol®. Polyoxyethylene-sorbitan-fatty acid esters (also called polysorbates), e.g., of from 4 to 25 alkylene moieties, for example monolauryl, trilauryl, palmityl, stearyl, and oleyl esters, including, for example, Tween®.

A group of suitable surfactants includes propylene glycol monocaprylate, mixtures of glycerol and polyethylene glycol esters of long fatty acids, polyethoxylated castor oils, nonylphenol ethoxylates (Tergitol®), glycerol esters, oleoyl macrogol glycerides, propylene glycol monolaurate, propylene glycol dicaprylate/dicaprate, polyethylene-polypropylene glycol copolymer, and polyoxyethylene sorbitan monooleate.

Hydrophilic solvents which may be used include, but are not limited to, alcohols, e.g., a water miscible alcohol such as absolute ethanol, and/or glycerol. Other alcohols include glycols, e.g., any glycol obtainable from an oxide such as ethylene oxide, e.g., 1,2-propylene glycol. Other non-limiting examples include polyols, e.g., a polyalkylene glycol, e.g., poly(C₂₋₃)alkylene glycol. One non-limiting example is a polyethylene glycol. The hydrophilic component may comprise an N-alkylpyrollidone, such as, but not; limited to, N—(C₁₋₁₄alkyl)pyrollidone, e.g., N-methylpyrollidone,tri(C₁-₄alkyl)citrate, e.g., triethylcitrate, dimethylisosorbide, (Cscl 3) alkanoic acid, e.g., caprylic acid and/or propylene carbonate. The hydrophilic solvent may comprise a main or sole component, e.g., an alcohol, e.g., C₁₋₄-alcohol, e.g., ethanol, or alternatively a component, e.g., which may be chosen from partial lower ethers or lower alkanols. Suitable partial ethers include, for example, Transcutol® (which has the formula C2Hs-[O—(CH2)2]2-0H), Glycofurol® (also known as tetrahydrofurfuryl alcohol polyethylene glycol ether), or lower alkanols such as ethanol, such as, for example, glycerol acetates and acetylated glycerol fatty acid esters.

In at least one embodiment of the present disclosure, the capsules are seamless and comprise a polysaccharide gel membrane outer surface shell, and optionally a coating on said gel membrane. The polysaccharide gel membrane may be ionic. In some embodiments, the seamless capsules encapsulate an oily phase comprising at least one marine oil, water, and at least one surfactant. In some embodiments, the oily phase is an emulsion, such as an oil-in-water emulsion, a water-in-oil emulsion, or a water-in-oil-in-water emulsion. According to some embodiments of the present disclosure, the marine oil is present in an amount of at least 50% by weight of the emulsion, such as at least 55%, at least 60%, at least 65%, at least 70%, at least 75%, at least 80%, at least 85%, or even at least 90% by weight of said emulsion. In at least some embodiments, the seamless capsules do not comprise marmelo mucilage.

In some embodiments, the polysaccharide gel membrane further comprises one or more secondary film formers chosen from cellulose acetate phthalate, cellulose acetate succinate, methyl cellulose phthalate, ethylhydroxycellulose phthalate, polyvinylacetatephtalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, propylene glycol alginate, polyvinyl alcohol, carrageenans, pectins, chitosans, guar gum, gum acacia, sodium carboxymethylcellulose, hydroxypropylmethyl cellulose, hydroxypropylcellulose, methylcellulose, starches, and maltodextrins.

In some embodiments of the present disclosure, the polysaccharide gel membrane comprising the seamless capsules is an ionic gel membrane comprising at least one of alginate, propylene glycol alginate, and pectin. Said at least one of alginate, propylene glycol alginate, and pectin may be present in the form of a pharmaceutically-acceptable salt, non-limiting examples of which include salts of calcium, strontium, barium, or aluminum. The ionic polysaccharide of the seamless capsules presently disclosed may comprise an alginate having a weight-average molecular weight ranging from about 20,000 Daltons to about 500,000 Daltons, such as from about 50,000 Daltons to about 500,000 Daltons, or about 100,000 Daltons to about 500,000 Daltons, or about 150,000 Daltons to about 500,000 Daltons, or about 150,000 Daltons to about 300,000 Daltons, or about 20,000 Daltons to about 200,000 Daltons, or from about 20,000 Daltons to about 100,000 Daltons, or from about 30,000 Daltons to about 80,000 Daltons, or from about 30,000 Daltons to about 60,000 Daltons, or even ranging from about 30,000 Daltons to about 40,000 Daltons. In some embodiments of the present disclosure, the ionic polysaccharide comprises a mixture of two alginate components, such as a mixture of (i) an alginate having a weight-average molecular weight ranging from about 30,000 Daltons to about 40,000 Daltons; and (ii) an alginate having a weight-average molecular weight ranging from about 150,000 Daltons to about 500,000 Daltons. In such embodiments, the ratio of (i) to (ii), (i):(ii), may range from about 0.1 to about 20, or about 1 to about 16.

The seamless capsules presently disclosed may be in a shape other than spherical. For example, in some embodiments of the present disclosure, the seamless capsules are oblong, oval, or cylindrical. The seamless capsules may be wet or dry.

The thickness of the polysaccharide gel film comprising the alginate shell of the seamless capsules presently disclosed may range from about 0.01 millimeter to about 50 millimeters. The polysaccharide gel film may be wet or dry. In some embodiments, the thickness of the polysaccharide gel film ranges from about 0.3 millimeters to about 4 millimeters. In some embodiments, the thickness of the polysaccharide gel film ranges from about 0.04 millimeters to about 0.5 millimeters.

The seamless capsules according to the present disclosure may have a wet capsule diameter ranging from about 0.5 millimeters to about 50 millimeters, such as about 1 millimeter to about 40 millimeters, wherein the gel membrane has a thickness ranging from about 0.1 millimeter to about 5 millimeters, such as about 0.3 millimeters to about 4 millimeters.

In some embodiments, the seamless capsule is dried, and the gel membrane is a dry polysaccharide gel film on the outer surface which constitutes up to 10% by weight of the dried seamless capsule. In some embodiments, the dry capsule has a diameter ranging from about 0.5 millimeters to about 35 millimeters, wherein the dry polysaccharide gel film has a thickness ranging from about 0.01 millimeters to about 5 millimeters. In some embodiments, the thickness of the dry polysaccharide gel film ranges from about 0.04 millimeters to about 0.5 millimeters.

According to the present disclosure, the omega-3 fatty acids may be administered to a subject in seamless capsules in a daily amount ranging from about 0.100 g to about 10.000 g, such as about 0.500 g to about 8.000 g, including from about 0.250 g to about 5.000 g and about 0.400 g to about 2.000 g. In the unit dosage form, the seamless capsules comprising omega-3 fatty acids may be present, for example, in an amount ranging from about 0.100 g to about 4.000 g, such as about 1.000 g to about 4.000 g, further such as 2.000 g and/or 4.000 g unit dosages. In at least one embodiment, the seamless capsules are administered to a subject in a unit dose ranging from about 0.400 g to about 2.000 g, such as about 0.400 g to about 1.740 g, such as about 0.420 g to about 1.680 g.

The daily dosage of omega-3 fatty acids can be administered in from 1 to 10 dosages, such as from 1 to 4 times a day. The administration may be oral or any other form of administration that provides a dosage of omega-3 fatty acid to a subject.

In one embodiment, the formulation(s) of the present invention may allow for improved effectiveness of active ingredients, with one or both administered as a conventional full-strength dose, as compared to the formulations in the prior art. In one embodiment, the formulation(s) of the present invention may allow for reduced dosages of omega-3 fatty acids as compared to the formulations in the prior art, while still maintaining or even improving upon the effectiveness of each active ingredient.

According to at least one embodiment, an oil-in-water emulsion is encapsulated in seamless capsules for oral administration. The seamless capsules may also be known generally as softgels.

Seamless capsules of the present disclosure may be prepared, for example, by a method disclosed in WO 2003/084516, comprising: (a) preparing an emulsion comprising oil, water, an emulsifier, and at least one of a water-soluble monovalent metal salt, polyvalent metal salt, and an acid, wherein the oil is present in an amount of at least 50% by weight of the emulsion; and (b) adding at least one portion of the emulsion to an aqueous gelling bath comprised of at least one ionic polysaccharide, thereby encapsulating the at least one portion of the emulsion in a polysaccharide gel membrane, and optionally (c) drying the resulting capsules.

In one embodiment of the present disclosure, the at least one polyvalent metal salt is calcium chloride (CaCl₂) and the at least one ionic polysaccharide is alginate. In one embodiment, the alginate is all or in part M-alginate. In one embodiment, the alginate is all or in part G-alginate. In one embodiment, the alginate is a mixture of M-alginate and G-alginate.

An advantage of having an omega 3 fatty acid oil and encapsulated dihydropyridine calcium blockers together in an alginate capsule, compared to a gelatin capsule, may be the opportunity to include an increased volume of the omega 3 fatty acids as active ingredients because the average film thickness of the seamless alginate capsule is significantly thinner, such as about 20% thinner, or 25% thinner, or even 30% thinner, than a gelatin film.

Thus, alginate capsules may have an increased fill volume which allows for a larger dosage per unit volume of the capsule. The fill volume of the capsule may increase by about 20%, or about 25%, or even about 30%, in comparison to gelatin capsules. Thus a fewer number of alginate capsules may be administered to a subject in order to achieve the same treatment, such as administration of 3 alginate capsules in place of 4 gelatin capsules. A smaller capsule can also be produced that has the same dosage as a larger gelatin capsule. The smaller size may increase patient compliance in that the capsules are more easily swallowed. The larger dosage per unit volume of capsule may decrease the number of capsules that would need to be taken to reach a given dose of the active pharmaceutical ingredient (API). According to the disclosure herein, API generally includes marine oil, such as fish oil, krill oil, and lipid compositions derived from fish, as well as omega-3 fatty acids comprising the marine oil. The seamless capsules presently disclosed may comprise other active pharmaceutical ingredients in addition to marine oil. The seamless capsules presently disclosed may be particularly suitable for large dose actives, acid-sensitive actives, or actives generating gastric irritation, or oxygen-sensitive actives.

A single seamless alginate capsule of the present disclosure may thus comprise less or more API than the amount of a gelatin capsule of the same size. For example, the seamless capsules presently disclosed may comprise about 0.5, 1, 1.5, or even 2 times the amount of API as compared to a gelatin capsule of the same size. In at least one embodiment a single seamless capsule comprises about 0.400 g to about 0.440 g of active pharmaceutical ingredient. In another embodiment, a single seamless capsule comprises about 0.800 g to about 0.880 g of active pharmaceutical ingredient. In yet another embodiment, a single seamless capsule comprises about 1.200 g to about 1.400 g of active pharmaceutical ingredient. In another embodiment, a single seamless capsule comprises about 1.680 g of active pharmaceutical ingredient. In another embodiment, a single seamless capsule comprises about 1.740 g of active pharmaceutical ingredient.

The preparation of the microcapsules may be carried out following any of the methods described in the literature. By way of description and without being limited thereto, the different processes of obtaining microcapsules could be grouped into the following categories:

### A) Simple Coacervation Method

A solution of the polymer and possible additives of the polymer in a suitable solvent is prepared. The drug to be encapsulated is suspended in said solution and a non-solvent of the polymer is added so as to force the deposit of the polymer on the drug crystals. Examples of such processes can be found in, for example, ES 2009346, EP 0 052 510, and EP 0346879.

### B) Complex Coacervation Method

Complex coacervation method is based on the interaction between two colloids that have opposite electric charges, which generates an insoluble complex that is deposited on the particles of the drug to be encapsulated, forming a membrane that will isolate the drug. Examples of such processes can be found, for example, in GB 1393805.

### C) Double Emulsion Method

The drug to be encapsulated is dissolved in water or in a solution of some other coadjuvant and is emulsified in a solution of polymer and additives in a suitable solvent, such as for example dichloromethane. The resulting emulsion is in turn emulsified in water or in an aqueous solution of an emulsifying agent, such as polyvinyl alcohol. Once this second emulsion is carried out the solvent in which the polymer and the plasticizer were dissolved in is eliminated by means of evaporation or extraction. The resulting microcapsules are obtained directly by filtration or evaporation. Examples of these processes can also be found in patent documents such as U.S. Pat. No. 4,652,441.

### D) Simple Emulsion Method

The drug to be encapsulated, the polymer, and the additives are dissolved together in a suitable solvent. This solution is emulsified in water or in an emulsifier solution, such as polyvinyl alcohol, and the organic solvent is eliminated by evaporation or by extraction. The resulting microcapsules are recovered by filtration or drying. Examples of these processes can also be found, for example, in U.S. Pat. No. 5,445,832.

### E) Solvent Evaporation Method

The drug to be encapsulated, the polymer, and additives are dissolved together in a suitable solvent. This solution is evaporated and the resulting residue is micronized to the suitable size. Examples of this process can also be found, for example, in GB 2,209,937.

The above methods may provide for continuous processing and flexibility of batch size. The capsules presently disclosed may be manufactured in low oxygen conditions to inhibit oxidation of the omega-3 fatty acids and/or additional active pharmaceutical ingredients during the manufacturing process.

The seamless capsules according to the present disclosure comprising omega-3 fatty acids may be administered to a subject for therapeutic treatment. The capsules may be administered to a subject to regulate at least one health problem, for example, irregular plasma lipid levels, cardiovascular functions, immune functions, visual functions, insulin action, neuronal development, hypertriglyceridemia, heart failure, and post myocardial infarction.

The following examples are intended to illustrate the present disclosure without, however, being limiting in nature. It is understood that the skilled artisan will envision additional embodiments of the invention consistent with the disclosure provided herein.

### EXAMPLES

### Example 1

### Capsule Preparation

An oil-in-water emulsion was prepared by combining:
Approximately 85% Lovaza™ (about 800-880 mg)
0.1-3% emulsifier by weight
0.1-6% CaCl2.2H2O (gelling salt) by weight
1-15% water by weight

The emulsion was extruded through a nozzle and cut into fragments, which were then dropped into a gelling bath. The gelling bath comprised 10-80% calcium alginate. The resulting capsules were washed in purified water and held in an aqueous plasticizer solution comprising 10-80% pharmaceutical grade glycerine. The capsules were then dried.

### Example 2

### Absorption

Bioaccessibility (potential availability for intestinal absorption) of n-3 fatty acids (EPA and DHA) in two alginate compositions (M-alginate and G-alginate) was studied for comparison with a gelatin formulation (Omacor). Experiments were performed under simulated fasting state conditions during transit through a dynamic gastrointestinal model of the stomach and small intestine. During the experiments, samples from different sites of the GI tract were taken in time to provide good insight on the (rate of) digestibility and kinetics of absorption of the nutrients or the stability and activity of functional ingredients.

The following compositions were tested:
(1) K85EE in gelatin capsules (Omacor®); 1000 mg;
(2) K85EE in M-alginate capsules (“high M”); 1000 mg;
(3) K85EE in G-alginate capsules (“high G”); 1000 mg.

Omacor® (composition 1) was commercially-available, and compositions (2) and (3) were prepared according to Example 1. The study was performed in a dynamic, multi-compartmental system of the stomach and small intestine simulating the successive dynamic conditions in the gastric-small-intestinal tract, such as body temperature, the pH curves, concentrations of electrolytes, and the activity of enzymes in the stomach and small intestine, the concentrations of bile salts in the different parts of the gut (for the production of micelles), and the kinetics of transit of the GI contents through the stomach and small intestine.

Experiments were performed under simulation of average physiological conditions in the upper gastrointestinal tract of healthy human adults during the fed state and the fasting state conditions. These conditions included especially the dynamics of gastric emptying and intestinal transit times, the gastric and the intestinal pH values, and the composition and activity of the secretion products. The formed micelles were filtrated continuously from the jejunum and ileum compartments of the model via hollow fiber semi-permeable membrane systems.

A specific filtration system was used to remove products of lipid digestion and lipophilic compounds that are incorporated in micelles. The removed material was collected to determine the bio-accessible fraction of fatty acids, cholesterol and fat soluble nutrients/compounds.

Under the fasted state conditions, the release and bioaccessibility of EPA and DHA from all three types of capsules was very low. Under fed-state conditions with a meal the bioaccessibility was increased for both gelatin and M-alginate capsules. For gelatin capsules the emptying of EPA and DHA out of the stomach was more efficient with a meal than without a meal.

The M-alginate capsules did not open at the same time in the simulations as in a phosphate buffer. For G-alginate capsules, EPA and DHA did not release and become bioaccessible during passage through the upper GI tract under fast or fed-state conditions. Phosphate was likely involved in dissociation of the alginate capsules; simulated electrolyte solutions did not contain phosphate. In the GI tract, phosphate mainly derives from the meal, with small amounts coming from the pancreas and bile secretion.

### Example 3

### Single-Dose Pharmacokinetics

Bioavailability of the compositions presently disclosed was studied in an animal (minipig; 5-6 months old) model representative of the human digestive system. The animals were orally dosed at two dose levels: 2 g (=2 capsules; “low dose”) and 4 g (=4 capsules; “high dose”). First all animals received 2 g of Omacor, followed in the next week by 2 g of K85EE alginate capsules (composition 2 as described in Example 1). This was subsequently repeated for the high dose groups (4 g) in the third and fourth week. Blood collection took place at pre-dose, 1, 2, 4, 6, 8, 10, 12, 16, 24, and 36 weeks after dosing.

In each plasma sample the EPA and DHA concentrations were determined as well as cholesterol, triglycerides and HDL levels. An additional set of parameters were determined at pre-dose and 24 h after dosing in the high dose groups; i.e., platelet count (Plt), alanine aminotransferase (ALAT), aspartate aminotransferase (ASAT), bilirubin (Tbil), prothrombine time (PTT), fibrogen (Fib), and activated partial thromboplastine time (APTT). Pharmacokinetic analysis was performed for EPA and DHA, where the data allowed the following parameter were calculated: maximum reached plasma concentration (Cₘₐₓ), time to reach the maximum concentration after dosing (Tₘₐₓ), the terminal half-life (T_{1/2},), the volume of distribution (V_{z}), the total clearance (Cl_{T}), the area under the concentration-time curve extrapolated to infinity (AUC_{0-∞}) and the area under the concentration-time curve extrapolated to the last measured time period (AUC₀₋ₜₙ).

In the low dose group, the K85EE alginate capsules showed a higher uptake of EPA and DHA in comparison with Omacor. For EPA, the Cₘₐₓ of the K85EE alginate capsules was 27.7 mg/L and for Omacor 22.3 mg/L. The Tₘₐₓ was later for the K85EE alginate capsules than for Omacor, i.e., 21 hours versus 9.5 h, respectively. For DHA the Cₘₐₓ of the K85EE alginate capsules was 18.6 mg/L and for Omacor 14.1 mg/L. The Tₘₐₓ between both formulations was similar (6.5 h). The AUC0-n for K85EE alginate was on average found to be 1.6 times higher for EPA in comparison with Omacor and 1.9 times higher for DHA.

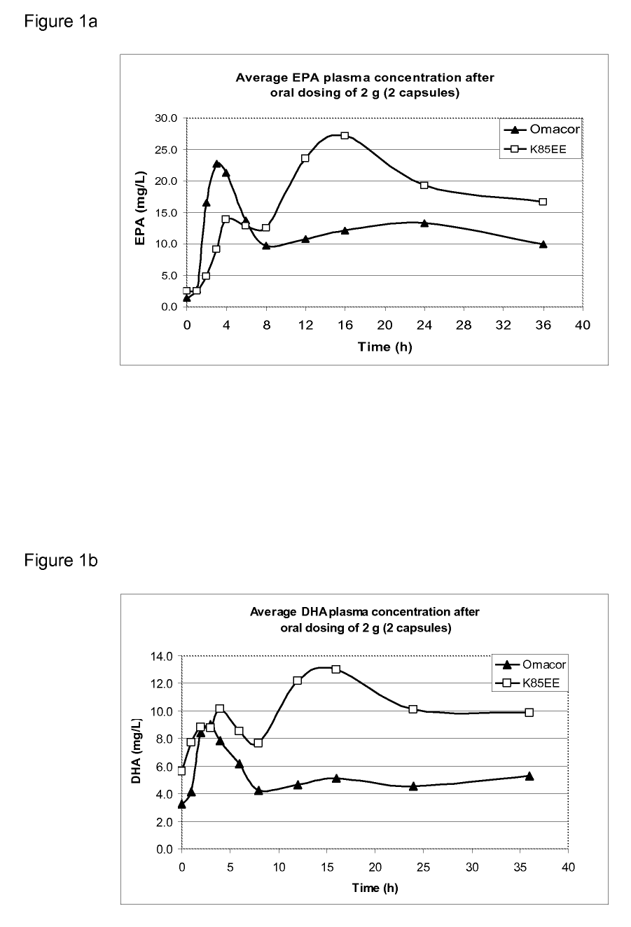

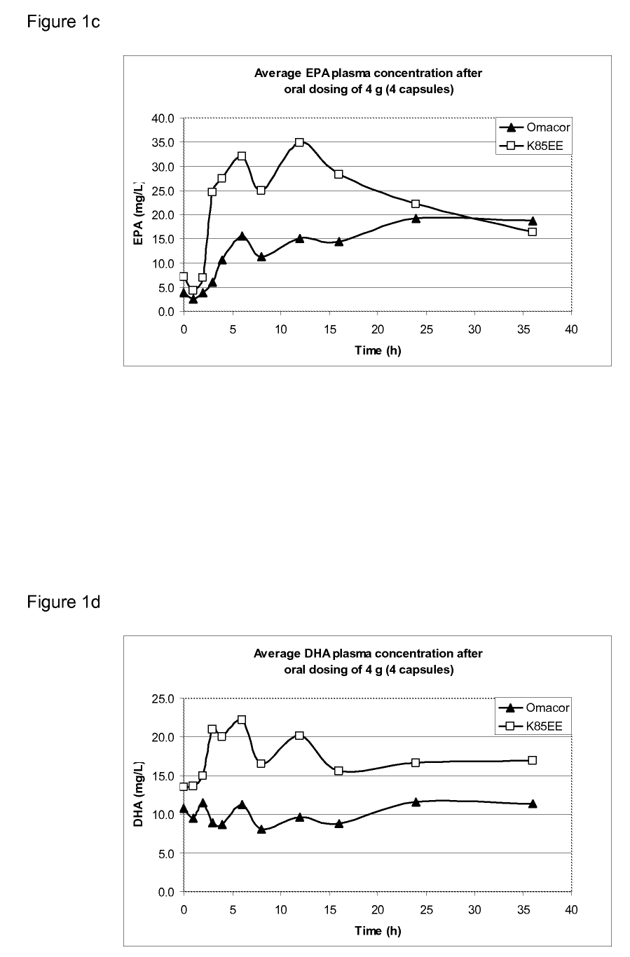

The high dose group also showed a higher uptake with the K85EE alginate capsules of EPA and DHA in comparison with Omacor. For EPA a Cₘₐₓ of the K85EE alginate capsules was 71.7 mg/L and for Omacor 25.53 mg/L. The Tₘₐₓ was earlier for the K85EE alginate capsules than for Omacor, i.e., 11.5 hours versus 23 h respectively. For DHA, the Cₘₐₓ of the K85EE alginate capsules was 42.4 mg/L and for Omacor 17.5 mg/L. The Tₘₐₓ for the K85EE alginate capsules was 4.5 h versus 17.5 h for Omacor. The AUC₀₋ₜₙ for K85EE alginate was on average found to be 1.5 times higher for EPA in comparison with Omacor and 1.7 times higher for DHA. Results appear in FIGS. 1a-d.

No statistical difference of the following parameters: Cₘₐₓ, Tₘₐₓ, AUC₀₋ₜₙ, AUC_{0-∞}, and T_{1/2} was found between the groups due the high variability between the animals within each dose group. After dosing of Omacor and the K85EE alginate capsules a decline was seen in all dose groups in the amount of cholesterol and HDL in plasma. The difference in triglycerides concentrations was less prominent.

The K85EE alginate capsules presented a higher bioavailability than Omacor in both dose groups. With 2 g, the bioavailability of EPA was around 1.6 times higher and, for DHA, 1.9 times higher in comparison with Omacor. If calculated on the geometrical means of the AUCs, the relative bioavailability of K85EE Alginate capsules was even higher, i.e., 2.5 times for both EPA and DHA in comparison with Omacor. With an oral dose of 4 g the bioavailability of EPA was 1.5 times higher and for DHA 1.7 times higher in comparison with Omacor.

The present data support an enhanced bioavailability of EPA and DHA from the K85EE alginate capsules as compared to Omacor.

### Example 4

### Unit Dose Administration

Seamless capsules are prepared according to the procedure of Example 1 for administration to a subject. The capsules are prepared in different unit dosages:

### Example 4 (a)

The active pharmaceutical ingredient (“API”) is EPA DHA present in ester or in acid form, wherein each single seamless capsule comprises about 0.400 g to about 0.440 g API. Thus, each capsule comprises about 0.5 times the amount of API of a comparative gelatin capsule.

### Example 4 (b)

The active pharmaceutical ingredient is EPA DHA present in ester or in acid form, wherein each single seamless capsules comprises about 0.800 g to about 0.880 g API. Thus each capsule comprises about the same amount (about 1 time the amount) of API of a comparative gelatin capsule.

### Example 4 (c)

The active pharmaceutical ingredient is EPA DHA present in ester or in acid form, wherein each single seamless capsules comprises about 1.200 g to about 1.400 g API. Thus each capsule comprises about 1.5 times the amount of API of a comparative gelatin capsule.

What is claimed is:

1. A seamless capsule comprising a polysaccharide gel membrane outer surface shell comprising at least one alginate wherein:
said outer surface encapsulates at least one emulsion comprising at least one oily phase,
said at least one oily phase comprises at least one marine oil and at least one surfactant,
said marine oil comprises at least 50% by weight of said emulsion, and
said emulsion does not comprise marmelo mucilage.

2. The capsule according to claim 1, wherein said at least one marine oil comprises at least 80% by weight of the emulsion.

3. The capsule according to claim 1, wherein the at least one marine oil comprises at least one omega-3 fatty acid chosen from eicosapentaenoic acid and docosahexaenoic acid.

4. The capsule according to claim 3, wherein at least one of said eicosapentaenoic acid and docosahexaenoic acid is in the form of free fatty acids, esters, or tri-glycerides.

5. The capsule according to claim 1, wherein said at least one marine oil is chosen from fish oils, krill oils, and lipid compositions derived from fish.

6. The capsule according to claim 3, wherein said at least one marine oil comprising at least one omega-3 fatty acid is chosen from Omacor®, Lovaza™, K85EE, K80EE, and AGP-103.

7. The capsule according to claim 1, wherein said at least one emulsion further comprises from about 0.1 to about 3% surfactant by weight and from about 0.1 to about 6% gelling salt by weight, each with respect to the total weight of said at least one emulsion.

8. The capsule according to claim 1, wherein said surfactant is chosen from glycerol acetates, glycerol fatty acid esters, acetylated glycerol fatty acid esters, propylene glycol esters, ethylene glycol esters, propylene glycol monocaprylate, mixtures of glycerol and polyethylene glycol esters of long fatty acids, polyethoxylated castor oils, nonylphenol ethoxylates, oleoyl macrogol glycerides, propylene glycol monolaurate, propylene glycol dicaprylate/dicaprate, polyethylene-polypropylene glycol copolymer, polyoxyethylene-sorbitan-fatty acid esters, and polyoxyethylene sorbitan monooleate.

9. The capsule according to claim 1, wherein said alginate is chosen from M-alginate, G-alginate, and a combination thereof.

10. The capsule according to claim 1, wherein the alginate comprises from about 1 to about 80% by weight with respect to the total weight of said shell.

11. The capsule according to claim 1, wherein said shell further comprises at least one additive chosen from coloring agents, stabilizers, sweetening agents, plasticizers, and hardeners.

12. The capsule according to claim 1, wherein said shell comprises from about 10 to about 80% plasticizer by weight with respect to the total shell weight.

13. The capsule according to claim 1, wherein the thickness of said shell ranges from about 0.01 mm to about 5 mm.

14. The capsule according to claim 1, wherein the thickness of said shell ranges from about 0.03 mm to about 1 mm.

15. The capsule according to claim 1, wherein the thickness of said shell ranges from about 0.2 mm to about 1.5 mm.

16. The capsule according to claim 1, wherein said at least one marine oil is present an amount ranging from about 0.400 g to about 0.440 g.

17. The capsule according to claim 1, wherein said at least one marine oil is present an amount ranging from about 0.800 g to about 0.880 g.

18. The capsule according to claim 1, wherein said at least one marine oil is present an amount ranging from about 1.200 g to about 1.400 g.

19. A seamless capsule comprising a polysaccharide gel membrane outer surface shell comprising at least one alginate, wherein said outer surface encapsulates at least one emulsion comprising at least one oil-in-water emulsion comprising:
about 85% of at least one marine oil by weight of said emulsion;
wherein said marine oil comprises about 90% omega-3 ethyl ester fatty acids by weight of said marine oil; and
wherein about 84% by weight of said omega-3 ethyl ester fatty acids comprise eicosapentaenoic acid ethyl ester and docosahexaenoic acid ethyl ester by weight of said omega-3 ethyl ester fatty acids;
about 0.1% to about 3% surfactant by weight of said emulsion;
about 0.1% to about 6% CaCl₂.2H₂O by weight of said emulsion; and
about 1% to about 15% water by weight of said emulsion.

20. A method of regulating at least one health problem chosen from irregular plasma lipid levels, cardiovascular functions, immune functions, visual functions, insulin action, neuronal development, hypertriglyceridemia, heart failure, and post myocardial infarction, comprising administering to a subject in thereof a seamless capsule comprising:
a polysaccharide gel membrane outer surface shell comprising at least one alginate, wherein:
said outer surface encapsulates at least one emulsion comprising at least one oily phase,
said at least one oily phase comprises at least one marine oil or marine oil composition and at least one surfactant,
said marine oil comprises at least 50% by weight of said emulsion, and
said emulsion does not comprise marmelo mucilage.

21. The method according to claim 20, wherein the health problem is chosen from hypertriglyceridemia, heart failure, and post myocardial infarction.

22. The method according to claim 20, wherein the capsule comprises a unit dose ranging from about 0.400 g to about 2.000 g.

23. The method according to claim 20, wherein the alginate comprising the outer surface shell of said capsule is M-alginate.

24. The method according to claim 20, wherein the capsule is administered once, twice or 3 times per day.

25. The method according to claim 20, wherein said capsule further comprises at least one other active pharmaceutical ingredient microencapsulated in said at least one marine oil or in said shell.

26. The method according to claim 20, wherein said at least one marine oil is chosen from fish oils, krill oils, and lipid compositions derived from fish.

27. The method according to claim 20, wherein said surfactant is chosen from glycerol acetates, glycerol fatty acid esters, acetylated glycerol fatty acid esters, propylene glycol esters, ethylene glycol esters, propylene glycol monocaprylate, mixtures of glycerol and polyethylene glycol esters of long fatty acids, polyethoxylated castor oils, nonylphenol ethoxylates, oleoyl macrogol glycerides, propylene glycol monolaurate, propylene glycol dicaprylate/dicaprate, polyethylene-polypropylene glycol copolymer, polyoxyethylene-sorbitan-fatty acid esters, and polyoxyethylene sorbitan monooleate.
